(19) United States
(12) Patent Application Publication
(10) Pub. No.: US 2019/0231382 A1
(43) Pub. Date: Aug. 1, 2019

(54) MEDICAL TREATMENT APPARATUS AND OPERATION METHOD FOR MEDICAL TREATMENT APPARATUS

(71) Applicant: OLYMPUS CORPORATION, Tokyo (JP)

(72) Inventor: Hikaru INOUE, Tokyo (JP)

(73) Assignee: OLYMPUS CORPORATION, Tokyo (JP)

(21) Appl. No.: 16/378,739

(22) Filed: Apr. 9, 2019

Related U.S. Application Data
(63) Continuation of application No. PCT/JP2016/082068, filed on Oct. 28, 2016.

(51) Int. Cl. A61B 17/32; A61B 18/14

(52) U.S. Cl. CPC A61B 17/320068; A61B 2018/00994; A61B 18/1442

(57) ABSTRACT

A medical treatment apparatus includes: a pair of holding members, each including a grasping surface configured to grasp a joining target site in living tissue; an energy application mechanism that is provided in at least one of the pair of holding members, the energy application mechanism being configured to apply joining energy and vibration energy to the joining target site through the grasping surface; and a control device configured to sense whether joining at the joining target site is completed by application of the joining energy to the joining target site, and control the energy application mechanism after the joining at the joining target site is completed to apply the vibration energy to the joining target site.

### CROSS-REFERENCE TO RELATED APPLICATION

This application is a continuation of PCT International Application No. PCT/JP2016/082068, filed on Oct. 28, 2016, the entire contents of which are incorporated herein by reference.

### BACKGROUND

The present disclosure relates to a medical treatment apparatus and an operation method for a medical treatment apparatus.

A medical treatment apparatus that applies energy to living tissue to treat (for example, coagulate or cut) the living tissue has been known (for example, International Publication Pamphlet No. WO 2010/076869).

The medical treatment apparatus (the ultrasonic and high-frequency operation system) described in Patent Literature 1 is configured to be capable of simultaneously applying both high-frequency energy and ultrasonic energy to living tissue from a treatment unit that contacts the living tissue. In order to reduce adhesion of living tissue to the treatment unit when the living tissue is treated, the medical treatment apparatus executes control to keep the amplitude of ultrasonic vibration within a given range.

### SUMMARY

A medical treatment apparatus according to one aspect of the present disclosure includes: a pair of holding members, each including a grasping surface configured to grasp a joining target site in living tissue; an energy application mechanism that is provided in at least one of the pair of holding members, the energy application mechanism being configured to apply joining energy and vibration energy to the joining target site through the grasping surface; and a control device configured to sense whether joining at the joining target site is completed by application of the joining energy to the joining target site, and control the energy application mechanism after the joining at the joining target site is completed to apply the vibration energy to the joining target site.

An operation method for a medical treatment apparatus according to one aspect of the present disclosure includes: sensing, after a pair of holding members grasp a joining target site in living tissue, whether joining at the joining target site is completed by application of joining energy to the joining target site through at least one of gripping surfaces of the pair of holding members; and applying, after the joining at the joining target site is completed, vibration energy to the joining target site through at least one of the gripping surfaces of the pair of holding members.

The above and other features, advantages and technical and industrial significance of this invention will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a medical treatment apparatus according to Embodiment 1;

FIG. 2 is a diagram illustrating a configuration of a second energy application unit;

FIG. 3 is a block diagram illustrating a configuration of a control device;

FIG. 4 is a flowchart representing joining control performed by the control device;

FIG. 5 is a diagram representing behaviors of the impedance of a joining target site during joining control;

FIG. 6 is a time chart representing types of energy applied to a joining target site and compressive load applied to the joining target site during joining control;

FIG. 7 is a diagram illustrating a configuration of a second energy application unit according to Embodiment 2; and

FIG. 8 is a diagram illustrating a configuration of a second energy application unit according to Embodiment 3.

### DETAILED DESCRIPTION

With reference to the drawings, modes for carrying out the disclosure (hereinafter “Embodiments”) will be described. Embodiments to be described below do not limit the disclosure. In the descriptions of the drawings, the same components are denoted with the same reference numbers.

### Embodiment 1

### Schematic Configuration of Medical Treatment Apparatus

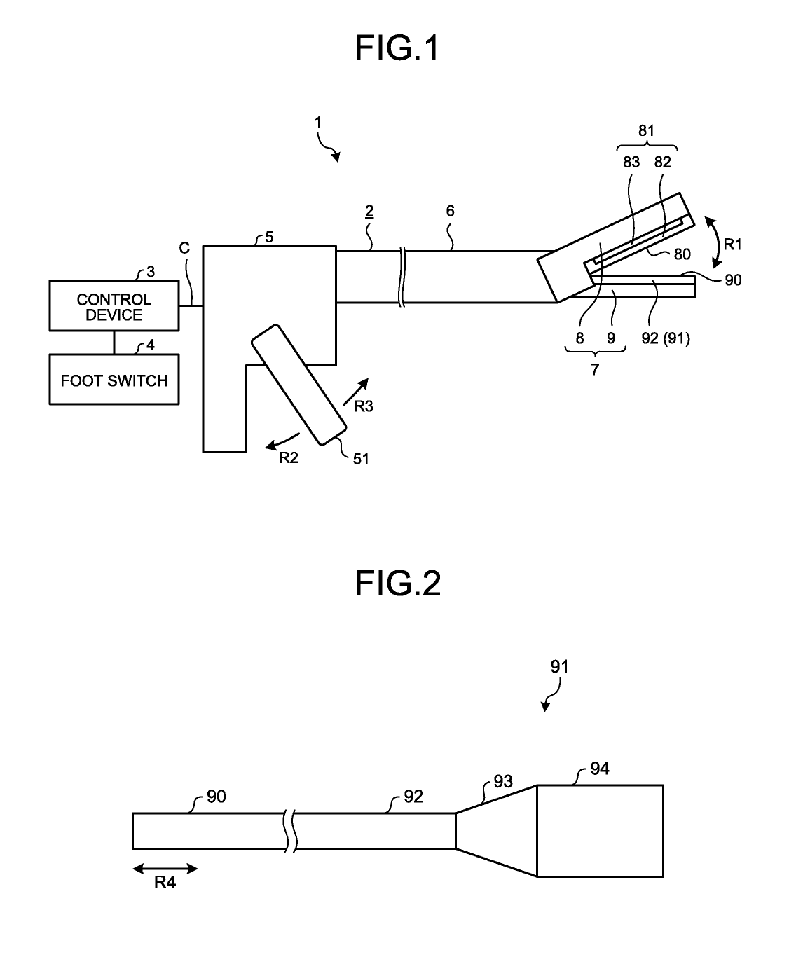

FIG. 1 is a diagram of a medical treatment apparatus 1 according to Embodiment 1.

The medical treatment apparatus 1 applies energy to a site (hereinafter “joining target site”) in living tissue on which treatment (joining (or anastomosing or sealing)) is to be given and thereby treats the joining target site. As illustrated in FIG. 1, the medical treatment apparatus 1 includes a treatment tool 2, a control device 3, and a foot switch 4.

### Configuration of Treatment Tool

The treatment tool 2 is, for example, a linear-type surgical medical treatment tool for treating the joining target site through the abdominal wall. As illustrated in FIG. 1, the treatment tool 2 includes a handle 5, a shaft 6, and a grasping unit 7.

The handle 5 is a part that an operator holds by hand. As illustrated in FIG. 1, an operation knob 51 is provided in the handle 5.

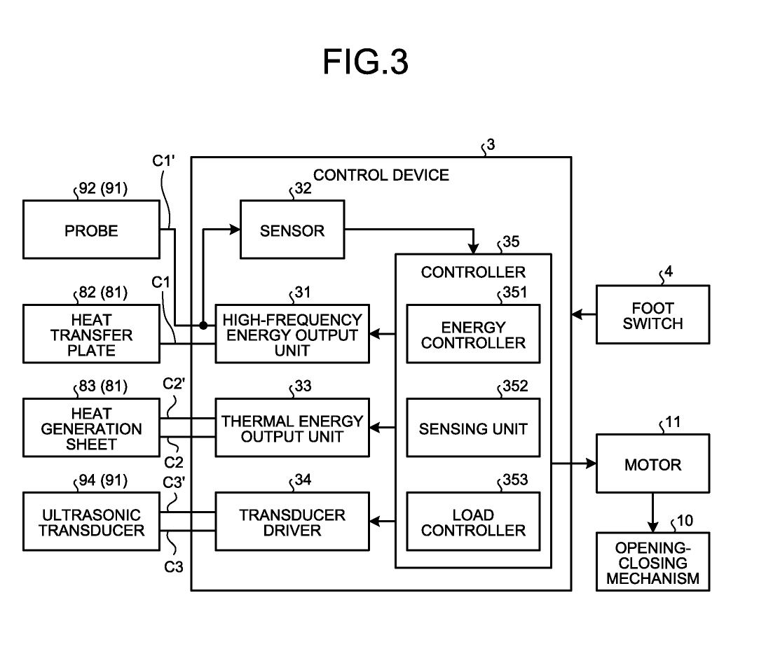

The shaft 6 has an approximately cylindrical shape and one end of the shaft 6 is connected to the handle 5 (FIG. 1). The grasping unit 7 is attached to the other end of the shaft 6. In the shaft 6, an opening-closing mechanism 10 is provided (see FIG. 3) to cause first and second holding members 8 and 9 (FIG. 1) of the grasping unit 7 to open or close in accordance with operation of the operator on the operation knob 51. In the handle 5, a motor 11 (see FIG. 3) connected to the opening-closing mechanism 10 is provided. While the first and second holding members 8 and 9 grasp the joining target site, the motor 11 causes the opening-closing mechanism 10 to operate under the control of the control device to change a compressive load applied to the joining target site from the first and second holding members 8 and 9. Furthermore, in the shaft 6, an electric cable C (FIG. 1) connected to the control device 3 is laid from one end to the other end via the handle 5.

### Configuration of Grasping Unit

The grasping unit 7 is a part that grasps the joining target site and treats the joining target site. As illustrated in FIG. 1, the grasping unit 7 includes the first holding member 8 and the second holding member 9.

The first and second holding members 8 and 9 are configured to be openable and closable in the direction of the arrow R1 (FIG. 1) (that is, capable of grasping the joining target site) in accordance with operation of the operator on the operation knob 51.

Specifically, as illustrated in FIG. 1, the first holding member 8 is pivotally supported on the other end of the shaft 6 such that the first holding member 8 is rotatable. On the other hand, the second holding member 9 is fixed to the other end of the shaft 6. In the Embodiment 1, the first holding member 8 is configured to be openable and closable with respect to the second holding member 9 according to operations of the operator on the operation knob 51. For example, when the operation knob 51 moves in the direction of the arrow R2 (FIG. 1), the first holding member 8 turns in a direction in which the first holding member 8 gets close to the second holding member 9. When the operation knob 51 moves in the direction of the arrow R3 (FIG. 1) that is opposite to the direction of the arrow R2, the first holding member 8 pivots in a direction in which the first holding member 8 separates from the second holding member 9.

The first holding member 8 is provided on the upper side with respect to the second holding member in FIG. 1. In the first holding member 8, as illustrated in FIG. 1, a first energy application unit 81 is provided on the surface of the first holding member 8, which is opposed to the second holding member 9.

The first energy application unit 81 has a function serving as the energy application mechanism according to the present disclosure. Under the control of the control device 3, the first energy application unit 81 applies a joining energy to the joining target site.

In the Embodiment 1, high-frequency energy and thermal energy are employed as joining energy to be applied to the joining target site.

As illustrated in FIG. 1, the first energy application unit 81 includes a heat transfer plate 82 and a heat generation sheet 83. The heat generation sheet 83 and the heat transfer plate 82 are layered in this order on the surface of the first holding member 8, which is opposed to the second holding member 9.

The heat transfer plate 82 is formed of a thin plate of copper, for example.

In the heat transfer plate 82, the plate surface on the lower side in FIG. 10 functions as a grasping surface 80 (FIG. 1) to grasp the joining target site between the grasping surface 80 and the second holding member 9.

The heat transfer plate 82 transmits heat from the heat generation sheet 83 to the joining target site through the grasping surface 80 (that is, applies thermal energy to the joining target site). The heat transfer plate 82 is joined to a lead line C1 (see FIG. 3) of the electric cable C. The heat transfer plate 82 applies high-frequency energy to the joining target site by being supplied with high-frequency power to the heat transfer plate 82 and a probe 92 described later via the high-frequency lead lines C1 and C1′ (see FIG. 3) by the control device 3. Thus, the heat transfer plate 82 also functions as a high-frequency electrode.

The heat generation sheet 83 is, for example, a sheet heater (resistor heater) and functions as a heat generator. Although detailed illustration is omitted, the heat generation sheet 83 has a configuration in which a resistor pattern is formed by vapor deposition, or the like, on a sheet substrate that is formed of an insulating material such as polyimide.

The resistor pattern is, for example, formed in U-shape that follows a shape of the outer edge of the heat generation sheet 83. The heat generation lead lines C2 and C2′ (see FIG. 3) of the electric cable C are joined respectively to both ends of the resistor pattern. The resistor pattern generates heat by being supplied with voltage (electric conduction) via the generation lead lines C2 and C2′ by the control device 3.

Although illustration is omitted in FIG. 1, an adhesive sheet for sticking the heat transfer plate 82 and the heat generation sheet 83 to each other is between the heat transfer plate 82 and the heat generation sheet 83. The adhesive sheet has a high heat transfer coefficient, is resistant to high temperatures, and has adhesiveness, and the adhesive sheet is, for example, formed by mixing ceramic with a heat transfer coefficient, such as alumina or aluminum nitride, with epoxy resin.

As illustrated in FIG. 1, a second energy application unit 91 is provided on a surface of the second holding member 9, which is opposed to the first holding member 8.

FIG. 2 is a diagram illustrating the second energy application unit 91.

The second energy application unit 91 has a function serving as the energy application mechanism according to the present disclosure. Under the control of the control device 3, the second energy application unit 91 applies vibration energy to the joining target site.

In the Embodiment 1, ultrasonic energy is used as the vibration energy to be applied to the joining target site.

As illustrated in FIG. 1 or FIG. 2, the second energy application unit 91 includes, the probe 92, a vibration enhancement member 93 (FIG. 2), and an ultrasonic transducer 94 (FIG. 2).

The probe 92 is formed of a conductive material and has an approximately cylindrical shape that extends along the axial direction of the shaft 6. The probe 92 is inserted into the shaft 6 such that its one end side (the right end side in FIG. 1) is exposed to the outside. The outer circumferential surface of the one end side functions as a grasping surface 90 (FIGS. 1 and 2) to grasp the joining target site between the grasping surface 90 and the heat transfer plate 82 (the grasping surface 80).

The probe 92 transfers ultrasonic vibration generated by the ultrasonic transducer 94 to the joining target site though the grasping surface 90 (that is, applies ultrasonic energy to the joining target site). The probe 92 is joined to the high-frequency lead line C1′ (see FIG. 3) of the electric cable C. The probe 92 applies high-frequency energy to the joining target site by being supplied with high-frequency power to the heat transfer plate 82 and the probe 92 via the high-frequency lead lines C1 and C1′ by the control device 3. Thus, the probe 92 also functions as a high-frequency electrode.

The vibration enhancement member 93 is attached to the other end (the right end in FIG. 2) of the probe 92 and is formed of a horn, or the like, that enhances the ultrasonic vibration generated by the ultrasonic transducer 94.

The ultrasonic transducer 94 is, for example, formed of a piezoelectric transducer using a piezoelectric element that expands and contracts according to application of alternating voltage and is connected to the probe 92 via the vibration enhancement member 93. Ultrasonic lead lines C3 and C3′ (see FIG. 3) of the electric cable C is joined to the ultrasonic transducer 94 and, under the control of the control device 3, alternating voltage is applied and accordingly the ultrasonic transducer 94 generates ultrasonic vibration.

In the Embodiment 1, the vibration enhancement member 93 and the ultrasonic transducer 94 are provided in a connected manner in an axial direction of the probe 92 as illustrated in FIG. 2. Therefore, longitudinal vibration (vibration in the axial direction of the probe 92) occurs according to ultrasonic vibration that occurs in the ultrasonic transducer 94. The one end side of the probe 92 (the left end side in FIG. 2) vibrates in the direction of the arrow R4 (FIG. 2). In other words, in the Embodiment 1, the second energy application unit 91 applies vibration energy (ultrasonic energy) of vibration in the in-plane direction (longitudinal direction) of the grasping surface 90 to the joining target site.

### Configurations of Control Device and Foot Switch

FIG. 3 is a block diagram of a configuration of the control device 3.

FIG. 3 mainly illustrates the relevant part of the disclosure as the configuration of the control device 3.

The foot switch 4 is a part that the operator operates with his/her feet and, according to the operation (ON), outputs an operation signal to the control device 3. According to the operation signal, the control device 3 starts joining control, which will be described below.

The unit to start the joining control is not limited to the foot switch 4. Alternatively, a switch with hand operation may be used.

The control device 3 overall controls operations of the treatment tool 2. As illustrated in FIG. 3, the control device 3 includes a high-frequency energy output unit 31, a sensor 32, a thermal energy output unit 33, a transducer driver 34, and a controller 35.

Under the control of the controller 35, the high-frequency energy output unit 31 supplies high-frequency power between the heat transfer plate 82 and the probe 92 via the high-frequency lead lines C1 and C1′.

The sensor 32 detects a voltage value and a current value that are supplied from the high-frequency energy output unit 31 to the heat transfer plate 82 and the probe 92. The sensor 32 outputs a signal corresponding to the detected voltage value and current value to the controller 35.

Under the control of the controller 35, the thermal energy output unit 33 applies voltage to the heat generation sheet 83 (electric conduction) via the heat generation lead lines C2 and C2′.

Under the control of the controller 35, the transducer driver 34 applies alternating voltage to the ultrasonic transducer 94 via the ultrasonic lead lines C3 and C3′.

The controller 35 includes a central processing unit (CPU), etc., and, when the foot switch 4 is on, executes joining control according to a given control program. As illustrated in FIG. 3, the controller 35 includes an energy controller 351, a sensing unit 352, and a load controller 353.

According to the operation signal from the foot switch 4 and a result of sensing performed by the sensing unit 352, the energy controller 351 controls operations of the high-frequency energy output unit 31, the thermal energy output unit 33, and the transducer driver 34. In other words, the energy controller 351 controls timing of application of high-frequency energy, thermal energy, and ultrasonic energy to the joining target site from the first and second energy application units 81 and 91.

The sensing unit 352 calculates an impedance of the joining target site based on the voltage value and the current value that are detected by the sensor 32. The sensing unit 352 sequentially compares the calculated impedance with first to third thresholds V1 to V3 and senses timing of application of high-frequency energy, thermal energy, and ultrasonic energy.

The load controller 353 causes the motor 11 to operate according to the operation signal from the foot switch 4 and the result of sensing performed by the sensing unit 352 and changes the compressive load (force to grasp the joining target site by the first and second holding members 8 and 9) that is applied to the joining target site from the first and second holding members 8 and 9.

### Operations of Medical Treatment Apparatus

Operations of the above-described medical treatment apparatus 1 will be described.

Joining Control performed by the control device 3 will be mainly described below.

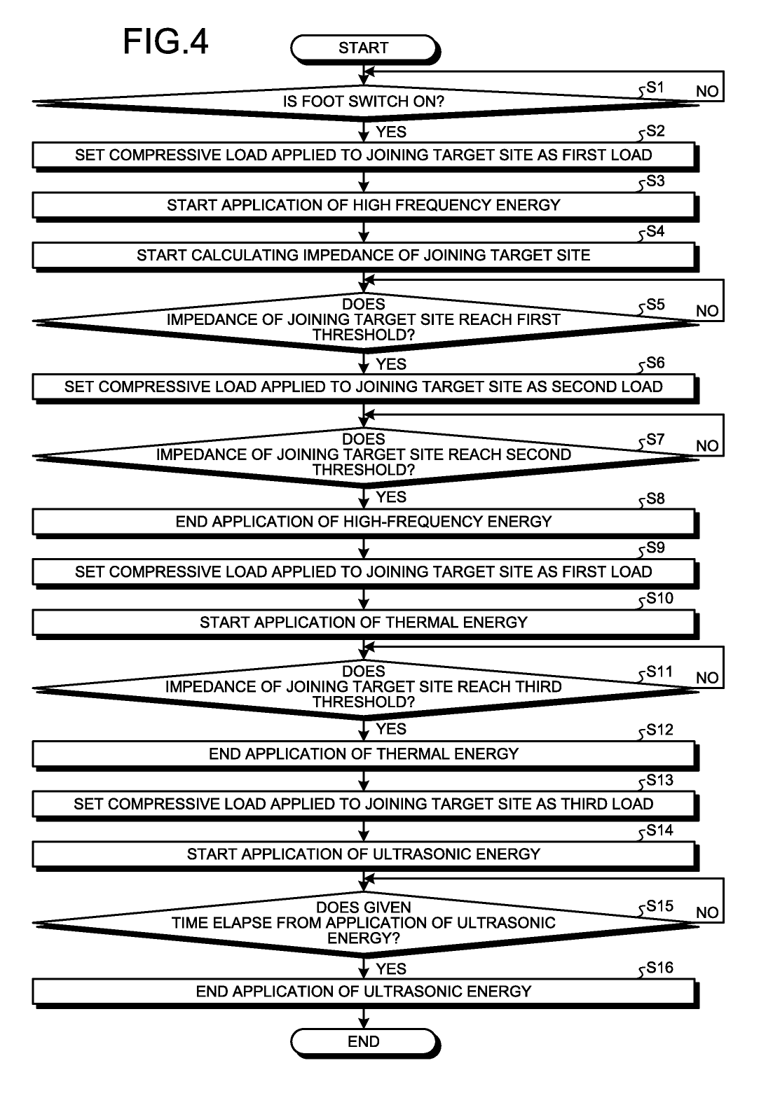

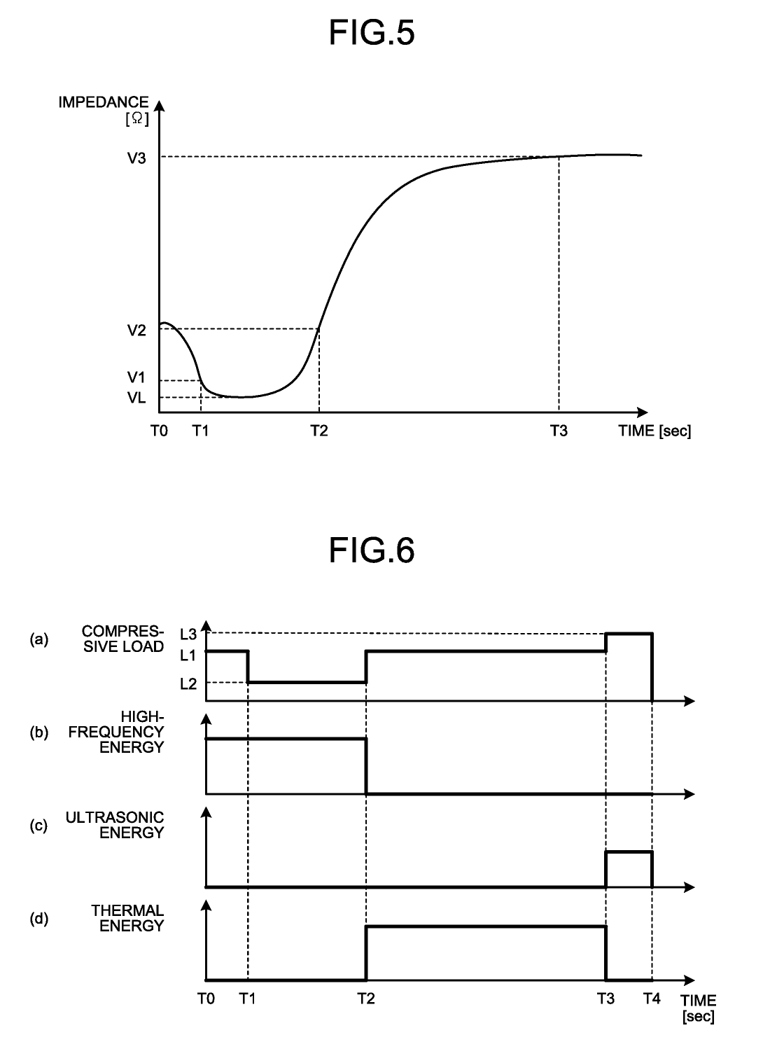

FIG. 4 is a flowchart of joining control performed by the control device 3. FIG. 5 is a diagram of behaviors of the impedance of the joining target site during joining control. FIG. 6 is a time chart representing types of energy applied to the joining target site and compressive load applied to the joining target site during joining control. Specifically, (a) to (d) of FIG. 6 represent time charts of compressive load, high-frequency energy, ultrasonic energy, and thermal energy, respectively.

The operator holds the treatment tool 2 by hand and inserts the tip of the treatment tool 2 into the abdominal cavity through the abdominal wall with, for example, a trocar. The operator then operates the operation knob 51 to open close the first and second holding members 8 and 9 to grasp the joining target site with the first and second holding members 8 and 9.

The operator then operates the foot switch 4 (ON) to start joining control performed by the control device 3.

When an operation signal from the foot switch 4 is input to the load controller 353 (the foot switch 4 is turned ON) (step S1: YES), the load controller 353 causes the motor 11 to operate and sets, to a first load L1 ((a) in FIG. 6), a compressive load to be applied to the joining target site from the first and second holding members 8 and 9 (step S2).

The energy controller 351 drives the high-frequency energy output unit 31 to start supply of high-frequency power from the high-frequency energy output unit 31 to the heat transfer plate 82 and the probe 92 (start application of high-frequency energy to a joining target site) (step S3).

For convenience of explanation, FIG. 4 illustrates a procedure in which step S3 is executed after step S2; however, practically, step S2 and step S3 are executed at approximately the same timing (Time T0 (see FIGS. 5 and 6)).

After step S3, based on a voltage value and a current value that are detected by the sensor 32, the sensing unit 352 starts calculating an impedance of the joining target site (step S4).

By applying the high-frequency energy to the joining target site, the impedance of the joining target site behaves as represented in FIG. 5.

In an early time band after the start of application of high-frequency energy (Time T0 in FIG. 5), the impedance of the joining target site reduces gradually. This is because application of high-frequency energy causes membrane breakdown in the joining target site and the extracellular matrix is extracted from the joining target site. In other words, the early time band is a time band in which the extracellular matrix is extracted from the joining target site and thus viscosity of the joining target site lowers (the joining target site softens).

After the impedance of the joining target site reaches a minimum value VL (FIG. 5), the impedance of the joining target site gradually increases. This is because Joule heat acts on the joining target site because of application of high-frequency energy and the joining target site generates heat and accordingly the moisture in the joining target site reduces (evaporates). In other words, after the impedance of the joining target site reaches the minimum value VL, it is the time band in which the extracellular matrix is not extracted from the joining target site and heat generation causes evaporation of the moisture in the joining target site and accordingly the viscosity in the joining target site increases (the joining target site coagulates).

After step S4, the sensing unit 352 keeps monitoring whether the impedance of the joining target site reaches the first threshold V1 (FIG. 5).

Here, the first threshold V1 is preset to a value slightly higher than the minimum value VL.

When it is determined that the impedance of the joining target site reaches the first threshold V1 (step S5: YES), the load controller 353 causes the motor 11 to operate at the time point when the impedance reaches the first threshold V1 (Time T1 in FIGS. 5 and 6) and sets, to a second load L2, the compressive load to be applied from the first and second holding members 8 and 9 to the joining target site ((a) in FIG. 6) (step S6).

Here, the second load L2 is preset to a load lower than the first load L1.

After step S6, the sensing unit 352 keeps monitoring whether the impedance of the joining target site reaches the second threshold V2 (FIG. 5) (step S7).

Here, the second threshold V2 is preset to a value approximately equal to an initial value of the impedance of the joining target site (the impedance at Time T0).

When it is determined that the impedance of the joining target site reaches the second threshold V2 (step S7: YES), the energy controller 351 ends application of high-frequency energy to the joining target site at the time point when the impedance reaches the second threshold V2 (Time T2 in FIGS. 5 and 6) (step S8).

Although application of high-frequency energy for treating the joining target site is terminated at step S8, the energy controller 351 supplies high-frequency power with the minimum output to the heat transfer plate 82 and the probe 92 via the high-frequency energy output unit 31 in order to make it possible to calculate impedance of the joining target site even after step S8 (after Time T2).

The load controller 353 causes the motor 11 to operate and sets, to the first load L1, the compressive load to be applied from the first and second holding members 8 and 9 to the joining target site (step S9).

The energy controller 351 further drives the thermal energy output unit 33 to start application of voltage (electric conduction) from the thermal energy output unit 33 to the heat generation sheet 83 (start application of thermal energy to the joining target site) (step S10: joining energy application step).

For convenience of explanation, FIG. 4 represents a procedure in which steps S9 and S10 are executed sequentially after step S8; however, practically, steps S8 to S10 are executed at approximately the same timing (timing at which the impedance of the joining target site reaches the second threshold V2 (Time T2)).

After Time T2, the impedance of the joining target site keeps increasing and finally saturates as illustrated in FIG. 5. This is because application of thermal energy causes evaporation of moisture in the joining target site and accordingly the joining target site coagulates. Therefore, by determining whether the impedance of the joining target site saturates, it is possible to determine whether joining at the joining target site completes.

After step S10, the sensing unit 352 keeps monitoring whether the impedance of the joining target site reaches the third threshold V3 (FIG. 5) (step S11: sensing step).

Here, the third threshold V3 is preset to a value at which the impedance of the joining target site saturates. In other words, at step S11, the sensing unit 352 determines whether joining at the joining target site completes.

When it is determined that the impedance of the joining target site reaches the third threshold V3 (step S11: YES), the energy controller 351 ends application of thermal energy to the joining target site at the time point when the impedance reaches the third threshold V3 (Time T3 (FIGS. 5 and 6)) (step S12).

The load controller 353 causes the motor 11 to operate and sets, to a third load L3, a compressive load to be applied from the first and second holding members 8 and 9 to the joining target site (step S13).

Here, the third load L3 is preset to a load higher than the first load L1.

Meanwhile, when living tissue sticks to the grasping surfaces 80 and 90, it is assumed that the living tissue dehydrates and dries in a state of contacting the grasping surfaces 80 and 90, and thereby the living tissue mechanically sticks to the grasping surfaces 80 and 90 (anchor effect). On this assumption, it is considered that the living tissue mechanically sticking to the grasping surfaces 80 and 90 may be detached by vibrating the grasping surfaces 80 and 90 because the living tissue does not follow the vibration of the grasping surfaces 80 and 90.

In order to cause the living tissue sticking to the grasping surfaces 80 and 90 to detach from the grasping surfaces 80 and 90 before completing the joining at the joining target site, the energy controller 351 drives the transducer driver 34 to start applying alternative voltage to the ultrasonic transducer 94 from the transducer driver 34 (start applying ultrasonic energy to the target site) (step S14: vibration energy application step).

For convenience of explanation, although FIG. 4 illustrates a procedure in which steps S13 and S14 are executed sequentially after step S12, steps S12 to S14 are practically executed at approximately the same timing (Time T3).

After step S14, the energy controller 351 keeps monitoring whether a given time elapses from application of ultrasonic energy at step S14 (step S15).

When it is determined that the given time elapses (step S15: YES), the energy controller 351 ends application of ultrasonic energy to the joining target site at the time point when the given time elapses (Time T4 (FIG. 6)) (step S16).

The medical treatment apparatus 1 according to the Embodiment 1 described above produces the following effects.

After joining at the joining target site completes, the medical treatment apparatus 1 according to the Embodiment 1 applies ultrasonic energy to the joining target site.

Therefore, even when living tissue sticks to the grasping surfaces 80 and 90 while the living tissue is being treated, ultrasonic energy is applied to the joining target site after the living tissue is treated and therefore the living tissue sticking to the grasping surfaces 80 and 90 detaches from the grasping surfaces 80 and 90. In other words, the operator is not needed to detach the living tissue from the grasping surfaces 80 and 90 by him/herself after treating the living tissue.

Accordingly, the medical treatment apparatus 1 according to the Embodiment 1 produces an effect that the operator is not forced to perform extra operations, and thereby convenience can be improved.

The medical treatment apparatus 1 according to the Embodiment 1 increases the compressive load that is applied to the joining target site from the first and second holding members 8 and 9 when applying ultrasonic energy to the joining target site after completion of joining at the joining target site. Accordingly, ultrasonic vibration is effectively transmitted to the joining target site and thus it is possible to effectively detach the living tissue from the grasping surfaces 80 and 90.

### Embodiment 2

Embodiment 2 of the present disclosure will be described.

The same components as those of the Embodiment 1 are denoted with the same reference numbers as those in the Embodiment 1 and detailed descriptions thereof will be omitted or simplified.

In the above-described Embodiment 1, ultrasonic energy is employed as the vibration energy according to the present disclosure.

On the other hand, in the Embodiment 2, vibration energy caused by rotary drive of a motor is employed as the vibration energy according to the present disclosure. In a medical treatment apparatus according to the Embodiment 2, the configuration of the second energy application unit 91 is different from that of the medical treatment apparatus 1 described in the above-described Embodiment 1.

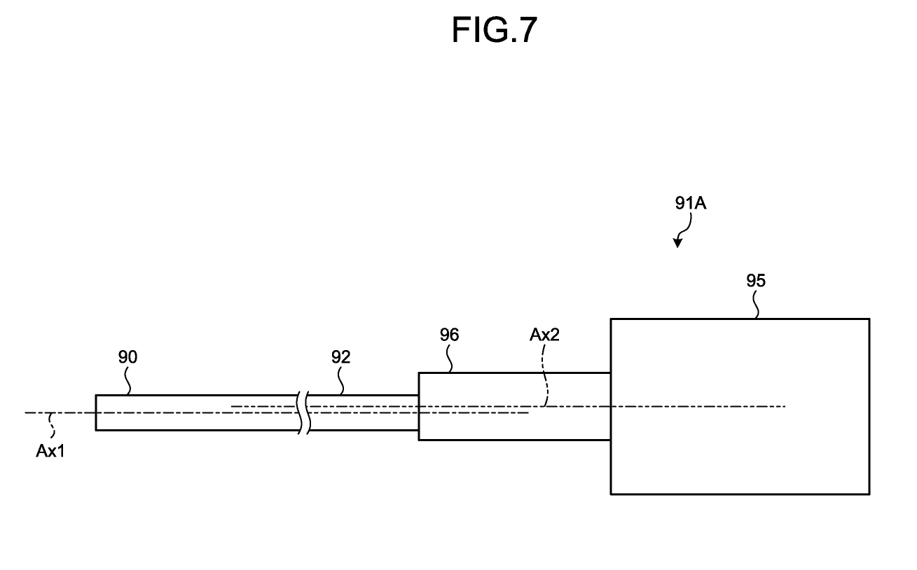

FIG. 7 is a diagram of a configuration of a second energy application unit 91A according to the Embodiment 2.

As illustrated in FIG. 7, the second energy application unit 91A according to the Embodiment 2 includes a motor 95 in addition to the probe 92 described in the Embodiment 1.

As illustrated in FIG. 7, the motor 95 is fixed inside the handle 5 (not illustrated in FIG. 7) in a posture such that the a center axis Ax1 of the probe 92 and a center axis Ax2 of a motor drive shaft 96 are parallel to each other. The other end of the probe 92 (right end part in FIG. 7) is fixed to the motor drive shaft 96 with the center axes Ax1 and Ax2 are slightly misaligned. When the motor 95 is driven and the motor drive shaft 96 rotates, the probe 92 vibrates while rotating about the center axis Ax2 because the probe 92 is eccentric to the motor drive shaft 96.

The energy controller 351 according to the Embodiment 2 applies vibration energy to the joining target site from the grasping surface 90 by driving the motor 95.

Even when the second energy application unit 91A using the motor 95 is employed as in the above-described Embodiment 2, the same effects as those of the above-described Embodiment 1 are produced.

### Embodiment 3

Embodiment 3 of the present disclosure will be described below.

In the following descriptions, the same components as those in the above-described Embodiment 1 will be denoted with the same reference numbers as those in the Embodiment 1 and detailed descriptions thereof will be omitted or simplified.

In the above-described Embodiment 1, the configuration to cause longitudinal vibration in the probe 92 is used as the second energy application unit 91.

On the other hand, in the Embodiment 3, a configuration to cause lateral vibration in the probe 92 is used as the second energy application unit. In other words, in the medical treatment apparatus according to the Embodiment 3, the configuration of the second energy application unit 91 is different from that of the medical treatment apparatus 1 described in the above-described Embodiment 1.

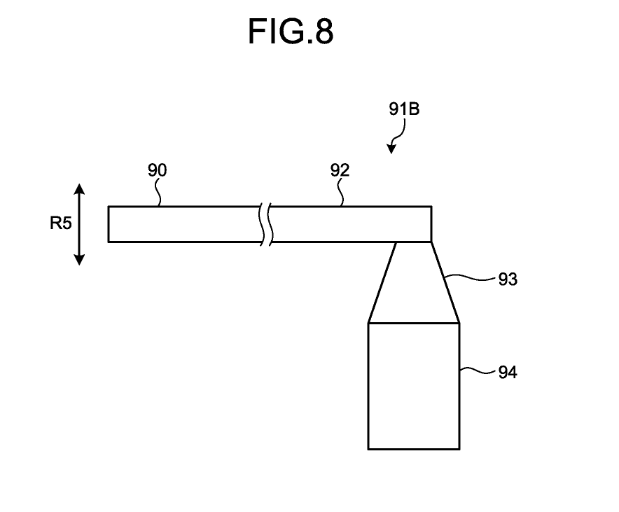

FIG. 8 is a diagram of a configuration of a second energy application unit 91B according to the Embodiment 3.

As illustrated in FIG. 8, the second energy application unit 91B according to the Embodiment 3 is different from the second energy application unit 91 described in the above-described Embodiment 1 in the state of connection of the probe 92 to the vibration enhancement member 93 and the ultrasonic transducer 94.

Specifically, as illustrated in FIG. 8, the vibration enhancement member 93 and the ultrasonic transducer 94 are attached to the outer circumferential surface of the other end (the right end in FIG. 8) of the probe 92. In the probe 92, lateral vibration (vibration in the radial direction of the probe 92) occurs according to the ultrasonic vibration that occurs in the ultrasonic transducer 94. The one end (left end side in FIG. 8) of the probe 92 vibrates in the direction of the arrow R5 (FIG. 8). In other words, in the Embodiment 3, the second energy application unit 91B applies vibration energy (ultrasonic energy) of vibration in an out-of-plane direction of the grasping surface 90 (the direction in which the grasping surfaces 80 and 90 are opposed to each other) to the joining target site. The amplitude of the vibration is set to amplitude that is equal to or larger than a surface roughness of the grasping surfaces 80 and 90 (for example, 10 μm) and that is smaller than a quarter of a distance between the grasping surfaces 80 and 90 in a state of grasping the joining target site.

The above-described Embodiment 3 produces the following effects in addition to the same effects as those of the Embodiment 1.

In the medical treatment apparatus according to the Embodiment 3, lateral vibration is caused in the probe 92 and the amplitude of the lateral vibration is set to the surface roughness of the grasping surfaces 80 and 90 or larger. Accordingly, it is possible to effectively detach living tissue mechanically sticking to the grasping surfaces 80 and 90 (anchor effect) from the grasping surfaces 80 and 90.

The joining target site is a site where two tissues overlap and the two tissues are joined between the grasping surfaces 80 and 90. For this reason, a half of the distance dimension between the grasping surfaces 80 and 90 in the state of grasping the joining target site corresponds to a thickness dimension of one of the tissues. When the amplitude of the lateral vibration is set to a half of the distance dimension or larger, the lateral vibration reaches the interface between the two tissues and thus it is difficult to keep sufficient joint strength.

In the medical treatment apparatus according to the Embodiment 3, the amplitude of the lateral vibration is set to amplitude smaller than a quarter of the distance dimension between the grasping surfaces 80 and 90 in the state of grasping the joining target site. Accordingly, the lateral vibration does not reach the interface between the two tissues forming the joining target site and thus it is possible to keep sufficient joint strength.

### Other Embodiments

While modes for carrying out the present disclosure have been described above, the present disclosure is not limited by the above-described Embodiments 1 to 3.

In the above-described Embodiments 1 to 3, the first energy application unit 81 is provided in the first holding member 8 and the second energy application unit 91 is provided in the second holding member 9. The configuration is not limited thereto and it suffices if a configuration enabling application of joining energy and vibration energy to the joining target site is used. For example, a configuration in which an energy application unit that applies joining energy and vibration energy is provided in only one of the first and second holding members 8 and 9 or a configuration in which energy application units each applying both joining energy and vibration energy are provided respectively in both the first and second holding members 8 and 9 may be employed.

In the Embodiments 1 to 3, two types of energy that are high-frequency energy and thermal energy are used as the joining energy according to the present disclosure. The joining energy is not limited thereto. Only one type of energy from high-frequency energy and thermal energy may serve as the joining energy according to the present disclosure or only ultrasound energy may serve as the joining energy according to the present disclosure. Alternatively, at least two types of energy from high-frequency energy, thermal energy and ultrasonic energy may be used as the joining energy according to the present disclosure. For example, in the above-described Embodiments 1 to 3, ultrasonic energy may be applied as joining energy to the joining target site between Time T1 and Time T2.

In the above-described Embodiments 1 to 3, the heat generation sheet 83 is used as a configuration to apply thermal energy to the joining target site. The configuration is not limited thereto. For example, a configuration in which a plurality of heat generation chips may be provided in the heat transfer plate 82 and electricity is conducted through the heat generation chips to transfer the heat of the heat generation chips to the joining target site via the heat transfer plate 82 may be used (for example, for the technology, see Japanese Unexamined Patent Application Publication No. 2013-106909).

In the Embodiments 1 to 3, timing of changing the compressive load applied to the joining target site and timing of starting and ending application of energy to the joining target site are adjusted based on the impedance of the joining target site. The adjustment is not limited thereto. For example, based on a pre-set time, physical property values, such as the temperature, thickness and hardness of the joining target site, or the impedance (ultrasonic impedance) of the ultrasonic transducer 94 during application of ultrasonic energy to the joining target site, the sensing unit may sense whether joining at the joining target site is completed to adjust the above-described timing.

In the above-described Embodiments 1 to 3, the flow of joining control is not limited to the order of processing according to the flowchart (FIG. 4) described in the above-described Embodiment 1 and may be changed within a range without inconsistency.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical treatment apparatus comprising:
a pair of holding members, each including a grasping surface configured to grasp a joining target site in living tissue;
an energy application mechanism that is provided in at least one of the pair of holding members, the energy application mechanism being configured to apply joining energy and vibration energy to the joining target site through the grasping surface; and
a control device configured to
sense whether joining at the joining target site is completed by application of the joining energy to the joining target site, and
control the energy application mechanism after the joining at the joining target site is completed to apply the vibration energy to the joining target site.

2. The medical treatment apparatus according to claim 1, wherein the energy application mechanism applies, to the joining target site, the vibration energy of vibration in an in-plane direction of the grasping surface.

3. The medical treatment apparatus according to claim 2, wherein the energy application mechanism applies, to the joining target site, the vibration energy of vibration in a longitudinal direction of the grasping surface.

4. The medical treatment apparatus according to claim 1, wherein
the energy application mechanism applies, to the joining target site, the vibration energy of vibration in an out-of-plane direction of the grasping surface, and
the vibration energy has amplitude equal to or larger than a surface roughness of the grasping surface.

5. The medical treatment apparatus according to claim 4, wherein the vibration energy has amplitude smaller than a quarter of a distance dimension between the grasping surfaces in a state of grasping the joining target site.

6. The medical treatment apparatus according to claim 1, wherein, when the joining target site is grasped by the pair of holding members, the control device switches a compressive load, which is applied to the joining target site from the pair of holding members, by increasing the compressive load after sensing that the joining at the joining target site is completed.

7. The medical treatment apparatus according to claim 1, wherein the energy application mechanism includes an ultrasonic transducer configured to generate ultrasonic vibration and apply the vibration energy to the joining target site by the ultrasound vibration.

8. The medical treatment apparatus according to claim 1, wherein the energy application mechanism includes a motor configured to apply the vibration energy to the joining target site by rotary drive.

9. An operation method for a medical treatment apparatus, the method comprising:
sensing, after a pair of holding members grasp a joining target site in living tissue, whether joining at the joining target site is completed by application of joining energy to the joining target site through at least one of gripping surfaces of the pair of holding members; and
applying, after the joining at the joining target site is completed, vibration energy to the joining target site through at least one of the gripping surfaces of the pair of holding members.
